(19) United States
(12) Patent Application Publication
(10) Pub. No.: US 2014/0079833 A1
(43) Pub. Date: Mar. 20, 2014

(54) FOOD PRODUCT COMPRISING RYE

(71) Applicants: Elin Ostman, Kavlinge (SE); Liza Rosen, Lund (SE); Inger Bjorck, Billinge (SE); Anne Nilsson, Rydeback (SE); Margareta Nyman, Lund (SE)

(72) Inventors: Elin Ostman, Kavlinge (SE); Liza Rosen, Lund (SE); Inger Bjorck, Billinge (SE); Anne Nilsson, Rydeback (SE); Margareta Nyman, Lund (SE)

(73) Assignee: RYEFACTOR AB, Flyinge (SE)

(21) Appl. No.: 14/115,538

(22) PCT Filed: May 4, 2012
(86) PCT No.: PCT/SE2012/050468

§ 371 Date: Nov. 4, 2013

(30) Foreign Application Priority Data
May 4, 2011 (SE) 1150393-5

(51) Int. Cl. A23L 1/30; A61K 36/899

(52) U.S. Cl. CPC A23L 1/3002; A61K 36/899
USPC 424/750; 426/18; 426/648

(57) ABSTRACT

A rye extract as well as food compositions including the extract. The use of the extract for the manufacture of a food composition, a dosage product, a pharmaceutical or a medicament. The extract is useful as a food composition, dosage product, pharmaceutical or medicament for the treatment, controlling or prevention of diseases or conditions related to metabolic syndrome, diabetes or obesity or in the promotion of satiety, weight loss or maintenance of desired body weight.

### TECHNICAL FIELD

The present invention concerns a rye extract as well as food compositions comprising said extract. The present invention also relates to the use of the extract for the manufacture of a food composition, a dosage product, a pharmaceutical or a medicament. The present invention further relates to the uses of said extract and food composition, dosage product, pharmaceutical or medicament for the treatment, controlling or prevention of diseases or conditions related to metabolic syndrome, diabetes or obesity or in the promotion of satiety, weight loss or maintenance of desired body weight.

### BACKGROUND

Obesity and diabetes are one of the fastest growing segments of unmet medical needs in the developed world. In the long term, obesity is associated with very serious consequences on health. For example, subjects with obesity are particularly at increased risk for chronic diseases such as heart disease, type 2 diabetes, high blood pressure, stroke, and some forms of cancer. Diabetes is associated with long-term complications that affect almost every part of the body. In order to avoid the personal distress caused by metabolic syndrome, diabetes and obesity, and also to limit the burden on medical service providers, there is an urgent need for treatments capable of controlling, e.g. treating or preventing or ameliorating, the symptoms and conditions associated with these disorders.

However, disturbances in the metabolic status can be prevented by changing the daily diet towards more whole grains, vegetables, legumes and dairy products. Also the dietary glycemic- and insulinemic indices of foods may play a role.

Treatments and effective nutritional approaches are highly sought after. Rye products are interesting in this context as they are usually consumed in wholegrain form and have been demonstrated to induce low insulin responses, with or without a simultaneous lowering of the glycaemic index (GI).

### SUMMARY OF INVENTION

It has been surprisingly found that addition of rye extracts of the invention to food compositions can improve the glycaemic response and lower acute insulin demand in a human being upon ingestion of said food composition, leading to lowered GI and/or improved glycaemic profile of said food composition and may thus be useful in controlling appetite, satiety and weight.

It has further been surprisingly found that the glycaemic profile of a food composition comprising said rye extract differed depending on the rye variety which has been used as the raw material of the rye extract.

Thus the present invention relates to rye extracts, wherein said rye extract comprises low and intermediate molecular weight indigestible carbohydrates (LIMWICs). The invention further relates to a supplemented extract wherein soluble viscous dietary fibre are added to said rye extracts.

The invention further relates to food compositions comprising said rye extract or said supplemented rye extract and to the uses of a rye extract and/or supplemented rye extract and food compositions of the invention.

### DEFINITIONS

For the purpose of this description, the following terms have the meanings ascribed to them. The term “extract” may refer to a liquid or dry product, such as for example a flour or disintegrate achieved by milling or a liquid homogenate.

The terms “Indigestible carbohydrates” (IC) and “dietary fibre”, (DF) are used interchangeably here.

The term “Indigestible carbohydrates” refers to carbohydrates which are normally present in the edible parts of plants, or similar carbohydrates, and which are resistant to digestion and absorption in the human small intestine. Indigestible carbohydrates may be either soluble or insoluble. Soluble indigestible carbohydrates undergo complete or partial fermentation in the large intestine. Some soluble indigestible carbohydrates have viscous properties and are here referred to as “Soluble viscous indigestible carbohydrates” or“soluble viscous dietary fibres”. Insoluble indigestible carbohydrates mostly add bulk and are fermented to a lesser degree.

The term “degree of polymerisation” (DP) refers to the number of monomeric units in a carbohydrate polymer.

The term “insulin-associated diseases or conditions” includes IRS, MS, IR, insulin sensitivity, IGT, low grade systemic inflammation and hyperinsulinemia as defined below. The term “Insulin Resistance Syndrome” (IRS) is used interchangeably with the term “Metabolic syndrome” (MS) and refers to a cluster of dysfunctions and metabolic risk factors which identifies individuals with increased risk of type 2 diabetes and cardio-vascular disease. IRS or MS may be characterized by at least two of the following abnormalities: insulin resistance, hyperinsulinemia, impaired glucose tolerance, hyperlipidemia, hypercholesterolemia, hypertension, and abdominal obesity.

The term “Insulin resistance” (IR) refers to a condition with impairment of insulin receptor signalling and a condition of impaired ability for glucose regulation.

The term “Insulin sensitivity” refers to a measure of degree of insulin action, with an insulin sensitive condition corresponding to a normal insulin receptor signalling and normal glucose metabolism.

The term “Impaired glucose tolerance (IGT)” refers to a pre-diabetic condition which is characterized by lowered insulin sensitivity in the fasting state, and/or post-prandial blood glucose responses above normal following a glucose challenge.

The term “Hyperinsulinemia” refers to a condition with elevated insulin levels.

The term “GI” refers to Glycemic Index, that is the post-prandial glycaemic response (incremental glycemic area under curve) to a carbohydrate test product expressed as a percentage of the corresponding response (incremental glycemic area under curve) with a carbohydrate equivalent amount of a reference product or pure glucose taken by the same subject. In the literature GI refers to a time period up to 1, 5 or 2 hours post meal. With a white wheat bread as reference product, GI values are approximately 38% higher than with pure glucose as reference. The GI values presented in the present application have been obtained using a white wheat bread as a reference product.

The term “Glycemic profile”, GP, is defined as the duration (min) for the incremental post-prandial glycemic response divided by the incremental glucose peak (iPeak, min/mM) elicited by a food. GP may be a better predictor of acute postprandial insulin demand, subjective rating of satiety in the late postprandial phase, and of second meal voluntary food intake than the GI (see Rosen et al, Nutrition Journal 2009).

The term “iPeak” means the glucose or insulin incremental peak and was calculated as maximum postprandial increase from baseline (fasting).

Calculating the GP of products makes it possible to distinguish a glycemic profile that has a low glucose iPeak but remains above fasting for a long time from that with a high glucose iPeak remaining above fasting for a short time. The latter is probably characterized by a larger hypoglycaemia. Such products may receive similar GI values, despite their different course of glycemia.

However, the GP value of a glycemic curve having a high incremental glucose iPeak, but remaining above fasting for a long time, will be similar to that of a product inducing a low glucose iPeak with short duration. In an attempt to discriminate also between these types of curves, the GP values were divided again with the glucose iPeak, thus giving the highest measured postprandial glucose concentration more weight in the equation. This duration/iPeak² quota has been named GP2.

The term “supplemented rye extract” refers specifically to a rye extract of the invention which has been supplemented by addition of one or more soluble viscous dietary fibres.

The terms Visello and Vicello refer to the same rye variety. Visello is a rye variety from KWS LOCHOW GMBH, Bergen, Germany. Breeder's reference: LPH 68. Picasso is a rye variety from Lochow-Petkus GmbH, Breeder's reference LPH 36.

The term “DP” means “Degree of polymerization”.

### BRIEF DESCRIPTION OF THE DRAWINGS

FIG. 1 shows glucose and insulin responses after the intake of breakfast products. Values are means±SEM, n=20 (n=19 for Kaskelott). Products not sharing the same letters were significantly different. Products not displaying letters were not different from any other test product, p<0.05 (ANCOVA, followed by Tukey's test)

1—WWB, 2—Commercial blend, 3—Amilo, 4—Evolo, 5—Picasso, 6—Vicello, 7—Kaskelott

FIG. 2 shows subjective satiety responses after the intake of breakfast products. Values are means±SEM, n=16 (n=15 for Kaskelott). Products not sharing the same letters were significantly different. Products not displaying letters were not different from any other test product, p<0.05 (ANCOVA, followed by Tukey's test). 1—WWB, 2—Commercial blend, 3—Amilo, 4—Evolo, 5—Picasso, 6—Vicello, 7—Kaskelott

### DETAILED DESCRIPTION OF INVENTION

Without wishing to be bound by theory, it is thought that the beneficial effects of the rye extract of the invention are derived from the rapid (within 3 hours of ingestion) fermentation of the carbohydrates found in said extract.

It is known that high molecular weight carbohydrate chains contribute to good baking qualities of flour and therefore the selection of rye varieties suitable for baking has also led to the selection for inclusion of such carbohydrate chains. However, as is shown, foods comprising said rye varieties suitable for baking may be low in or lack one or more of the carbohydrates comprised in the rye extract of the invention. This deficiency may be remedied by addition of the rye extract of the present invention. In this manner it becomes possible to lower the GI and/or improve the GP and/or GP2 of the foods to which the rye extract of the invention is added. Addition of the rye extract of the invention to conventional rye breads leads to maintained good baking results while lowering the GI/ and/or improving the GP and/or GP2 of the bread. The food compositions to which the rye extract of the invention has been added may be used in the treatment or prophylaxis of insulin-associated disorders, such as metabolic syndrome, insulin resistance, pre-diabetes symptoms and weight-loss, as well as weight maintenance.

Additionally, the data indicates that some rye varieties may be more insulin saving than others. Use of certain rye varieties presented below in the rye extract may yield more effective food supplements.

The rye extract of the invention may be characterised in that it comprises both a low molecular weight fraction of carbohydrates, (defined as the carbohydrates which remain in solution after enzymatic digestion of protein and starch followed by precipitation with 4 volumes 95% ethanol, reaching a final ethanol concentration of 78% (v/v) in the method described by Asp et al (1983 J Agric Food Chem); and a sub-fraction of carbohydrates which are precipitated out of solution by the ethanol precipitation in the same method.

Thus in one embodiment the present invention relates to a rye extract comprising one or more carbohydrates selected from raffinose, stachyose, fructans, arabinoxylans, arabinogalactans, beta-glucans and resistant starch.

One or more of said carbohydrates may for example have a degree of polymerisation of 3 to 300, such as 3 to 270, 3 to 250, 3 to 230, such as 200 to 250 such as 3 to 200, such as 3 to 220, such as 3 to 100, such as 3 to 50, such as 3 to 40, such as 3 to 30, such as 3 to 20, such as 10 to 80, 10 to 50, 20 to 60, or 20 to 80. In further examples said carbohydrates may have a degree of polymerisation of 3 to 10, or 10 to 300, such as 50 to 300, 100 to 300, 150 to 300, 100 to 200, or 50 to 150. The amount of carbohydrates in the extract may be analysed by liquid chromatography using AOAC Official Method 2001.03. The carbohydrate content and their degree of polymerisation may be determined by methods known in the art. DP distribution of fructan in the extract may be analysed as described by Rakha et al (Food Chemistry 2010). Amount of arabinoxylans and arabinogalactans as well as degree of substitution of arabinoxylans is determined as described by Delcour et al (1999 Food Chemistry) Raffinose and Stachyose may be determined using commercially available enzyme-based kits.

The rye extract of the invention may also comprise one or more of phenolic acids tocotrienols, stigmasterol, brassicasterol, campesterol, choline, betaine, alkylresorcinols, tocopherols, ferulic acid, sinapic acid, vanillic acid, caffeic acid, syringic acid, 4-hydroxy benzoic acid and phytic acid. Said phenolic acids may for example derive from the rye and/or may be exogenous and added to the extract.

The rye extract of the invention may also comprise one or more of Magnesium, Chromium, Calcium, Selenium and Zinc. These minerals may for example derive from the rye and/or may be exogenous and added to the extract.

The rye extract of the invention may consist of extract from whole rye plants, whole rye kernels, rye endosperm or combinations thereof.

The rye extract may be achieved by for example wet or dry processes, for example milling, grinding or homogenization. The rye extract may be in dry form, such as a powder, flour or granulate. The rye extract may alternatively be in liquid form, such as a liquid extract or homogenate. The liquid extracts or homogenates may be dried to yield a dry form of said rye extract exemplified above.

The invention in a further embodiment relates to a method of making the rye extract of the invention.

In one embodiment the method of the invention comprises the steps of

a.) providing a rye material, such as rye kernels and/or plant material from rye such as whole straw
b.) milling said rye material
c.) dispersing said milled rye material in a liquid;
wherein the liquid of step c.) may be one or more selected from the group consisting of liquids suitable for human consumption. Examples of such liquids are water, buffers such as phosphate buffer, and alcohols, such as ethanol, and combinations thereof.
d.) incubating said dispersion of step c.)
wherein step d.) may be performed at a temperature in the range from 4 and 100° C., such as 15° C. to 50° C., such as 18° C. to 25° C.; or such as about 20° C., about 21° C., or 30° C. to 55° C., such as 35° C. to 45° C.; or about 37° C.; or below 50° C., or below 47° C. The incubation may be for 15 minutes to 72 hours, such as for 1 hrs to 72 hours, such as 1 hr to 18 hours, such as 2 hrs to 18 hrs, 4 hrs to 18 hrs, 6 hrs to 18 hrs, 10 hrs to 12 hrs, such as 36 hrs, such as 48 hrs, such as 72 hrs; or for 1 hr to 12 hrs, such as 1 hr to 2 hrs, such as 3 to 4 hrs, such as 6 to 8 hrs. Step d.) may optionally be stirred.
e.) optionally solubilizing and/or disintegrating aggregates in the dispersion from step d.),
wherein step e.) may be achieved for example by sonication or any other suitable means;
f.) optionally heating the solubilized dispersion from step e.)
wherein the heating may be to a temperature of about 15° C. to about 100° C., such as about 60° C. to about 95° C., such as about 85° C. to about 95° C., such as about 95° C.; the heating may be performed with stirring.
g.) optionally adding an alcohol suitable for human consumption, such as ethanol to reach a concentration of from 20% to 100%, such as from 20% to 98%, such as from 30% to about 40%, or such as from about 40% to about 80%, or such as about 40%, or about 60%, or about 80%; and allowing precipitation to take place
h.) optionally filtering away the precipitate from step g.)
i.) recovering the liquid phase, for example by centrifuging and recovering the liquid supernatant
j.) optionally concentrating the liquid phase from i.)

In another embodiment the method of the invention comprises the steps of

a.) providing a rye material, such as rye kernels and/or plant material from rye such as whole straw
b.) milling said rye material
c.) dispersing said milled rye material in a liquid;
wherein the liquid of step c.) is selected from the group consisting of water, phosphate buffer and ethanol and combinations thereof
d.) incubation of said dispersion of step c.)
wherein step d.) is performed at a temperature in the range from 15° C. to 50° C.
e.) solubilizing and/or disintegrating aggregates the dispersion from step d,) by sonication;
f.) optionally heating the solubilized dispersion from step e.)
wherein the heating may be to a temperature of about 15° C. to about 100° C., such as about 60° C. to about 95° C., such as about 85° C. to about 95° C., such as about 95° C.; the heating may be performed with stirring.
g.) adding ethanol to reach a concentration of from about 40% to 80%, or such as about 40%, or about 60%, or about 80%; and allowing precipitation to take place
h.) optionally filtering away the precipitate from step g.)
i.) recovering the liquid phase by centrifuging and recovering the liquid supernatant
j.) optionally concentrating the liquid phase from i.)

In one embodiment the method of the invention comprises the steps of

a.) providing a rye material, such as rye kernels and/or plant material from rye such as whole straw
b.) milling said rye material
c.) dispersing said milled rye material in phosphate buffer,
k) a fermentation step where a probiotic is added to the dispersion of step c.)
d.) incubation of said dispersion of step c.) wherein step d.) is performed at a temperature in the range from 35° C. to 45° C. for 2 hrs to 18 hrs and is stirred.
e.) solubilizing and/or disintegrating aggregates the dispersion from step d.)
f.) optionally heating the solubilized dispersion from step e.)
g.) adding ethanol to reach a concentration of about 40% to about 80%, and allowing precipitation to take place
h.) optionally filtering away the precipitate from step g.)
i.) recovering the liquid phase, for example by centrifuging and recovering the liquid supernatant
j.) optionally concentrating the liquid phase from i.)

In one embodiment the method of the invention comprises the steps of

a. providing a rye material, such as rye kernels or whole straw
b. milling said rye material
c. dispersing said milled rye material in water or phosphate buffer
d. heating said dispersion to 37 degrees celsius and stirring for 48 hours
e. sonicating the dispersion from step d
f. heating the sonicated dispersion from step e to 95 degrees Celsius and stirring for 30 mins.
g. adding ethanol to reach a concentration of 60%
g allowing precipitation to take place
h. filtering away the precipitate from step g
i recovering the water phase
j. concentrating the water phase from i.

In alternative embodiments of the methods of the invention, a fermentation and/or incubation step k.) is inserted after step c.), wherein for example a yeast or sour dough, and/or enzymes such as xylanases, beta-glucanases, beta-mannases and/or one or more probiotic is added and the dispersion from step c.) is allowed to ferment or incubate. An example of such fermentation is the proofing of a bread dough comprising the dispersion of step d.). Step d.) may be adjusted according to the starting material to be of longer or shorter duration, as suitable. Thus, the invention also relates to a method wherein a fermentation step k.) is inserted after step c.), wherein one or more yeast and/or enzymes and/or probiotic is added.

Examples of suitable yeast and/or probiotics include yeasts such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostreptococcus, Bacillus, Pediococcus, Micrococcus, Leuconostoc, Weissella, Aerococcus, Oenococcus and Lactobacillus. Specific examples of probiotic microorganisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, Staphylococcus xylosus, Lactobacillus acidophilus, Lactobacillus thermofilus, Lactobacillus bulgaricus, Lactobacillus plantarum, Lactobacillus reuteri, Bifidobacterium bifidum, Bifidobacterium longum. Caseii, Lactobacillus iners.

For example, the probiotic microorganism may be selected from the group consisting of Lactobacillus acidophilus, Lactobacillus thermophilus, Lactobacillus bulgaricus, Lactobacillus plantarum, Lactobacillus reuteri, Bifidobacterium bifidum, Bifidobacterium longum. Caseii and Lactobacillus iners.

In another alternative embodiment of the method a protease digestion step 1.) is included, for example after step c.), wherein a protease solution is added and the dispersion incubated, for any period suitable to allow digestion, for example for 30 mins, such as for 2 hours, for example 3-4 hours.

In a further alternative the method of the invention comprises a step for separation according to size, such as for example precipitation with ethanol, wherein the ethanol may be used at an end concentration from 50 to 80%, such as for example 60%, 65%, 60-68%, 62%, 66%, 70%, 72%, 75%, 76%, 78%, 79% or 80%. As an alternative, other size separation methods may be used such as dialysis membranes or ultracentrifugation, or membrane filters.

In yet further embodiments of the method of the invention, different starting materials may be used. For example rye bread past due date, superfluous dough from bakeries, rye with a low falling number such as amylase-damaged rye with low falling number, or rye having less good baking characteristics are examples of various rye materials. Depending on the starting material steps adjustments to the method may be necessary, which will be apparent to the person skilled in the art.

The invention in one aspect relates to a rye extract obtainable by the methods according to the invention. In one embodiment the invention relates to a rye extract obtained by the methods according to the invention.

The invention relates in one aspect to an extract of rye which comprises Fructan (DP=1-10), Fructan (DP=10-20), Arabinoxylans (DP>10), Non-cellulosic beta-glucans (DP>100) and Raffinose (DP=3-5).

A further aspect of the invention relates to a rye material as a starting material for the method of the invention which comprises Fructan (DP 1-10), Fructan (DP=10-20), Arabinoxylans (DP>10), Non-cellulosic beta-glucans (DP>100) and Raffinose (DP=3-5), and does not comprise Arabinoxylan oligosaccharides (DP=2-10), mannoproteins, and Mellobiose (DP=2). In one embodiment said rye material comprises these said components in the same or similar amounts or ratios as presented in Example 4. Said rye material may be from rye variety Visello and/or Picasso, but may also be from other rye varieties. In one embodiment the starting material is from Visello and/or Picasso.

Said starting material may be employed in the methods of the invention, and be comprised in or constitute the starting material which is provided in step a.) of the methods of the invention.

In a further embodiment of the rye extract of the invention, the rye extract comprises or consists of the components Fructan (DP 1-10), Fructan (DP=10-20), Arabinoxylans (DP>10), Non-cellulosic beta-glucans (DP>100) and Raffinose (DP=3-5), as well as Arabinoxylan oligosachharides (DP=2-10), mannoproteins and Mellobiose (DP=2) (see Table 5).

In embodiments of rye extract of the invention, the rye extract may comprise or consist of said components in the following percentages.

Fructan (DP 1-10), may be present in from 2 to 8%, such as from 3 to 7%, such as from 3 to 4%, such as about 4%, such as 4%;
Fructan (DP=10-20) may be present in from 16 to 25%, such as from 18 to 24%, such as 18 to 23%, such as 19 to 23%;
Arabinoxylans (DP>10) may be present in from 0.5% to 10%, such as from 1 to 9%, such as 1 to 8%, such as 1.4 to 7.4%;
Arabinoxylan oligosaccharides (DP=2-10), may be present in from 6.4 to 7%; such as from 5 to 8%, such as 6 to 8%, such as 6 to 7%;
Non-cellulosic beta-glucans (DP>100) may be present in 7 to 12%, such as from 8 to 12%, such as 8%;
Mannoproteins, may be present in from 10 to 25%, such as 12 to 23%, such as 14 to 21%;
Raffinose (DP=3-5) may be present in from 9 to 15%, such as 10 to 15%, such as 11 to 14%;
Mellobiose may be present in from 15 to 32%, such as 18 to 30%, such as 20 to 29%, such as 21 to 28%.

Optionally, the extract may comprise additional components, for example a liquid acceptable for human consumption to 100%. If the rye extract also comprises additional components and/or is supplemented with soluble viscous dietary fibre, the ratio between said the components above is maintained. See also Table 5.

In another embodiment the extract consists of said components and the sum of the percentages of the components is 100%.

The invention further relates to the methods according to the invention, the rye extract of the invention including the supplemented rye extract of the invention, wherein the rye material is selected from the group consisting of rye variety Visello and Picasso or a combination thereof. The rye material may in another embodiment also be from other rye varieties which display the same or similar ratios of the above mentioned components (Fructan (DP=1-10), Fructan (DP=10-20), Arabinoxylans (DP>10), Non-cellulosic beta-glucans (DP>100) and Raffinose (DP=3-5)) as present in Visello and/or Picasso, and/or which display same or similar ratios of the components presented in Example 4.

In one embodiment the invention relates to a supplemented rye extract wherein soluble viscous dietary fibre has been added to the rye extract. In one example said soluble viscous dietary fibre is not from rye. The soluble viscous dietary fibre may for example be exogenous, ie from a source other than the rye extract. The supplemented rye extract may comprise soluble viscous dietary fibre from one or more sources such as for example oats, barley; algae, maize, sorghum, millet, quinoa and bacteria. Examples of said soluble viscous dietary fibre include for example one or more of agar, alginates; carubin; pectin; beta-glucan, such as oat beta-glucan, and barley beta glucan; carrageenans; furcellaran; psyllium, such as psyllium seed husk; mucilages and gums; alfalfa, clover, fenugreek, tamarind flour, pectin and its derivatives, scleroglucan, mannoglucans. Examples of gums are one or more of konjac gum, xanthan gum, guar gum (guaran gum), gum tragacanth, arabic gum, karaya gum, gum ghatti, gellan gum and other related sterculia gum. Said soluble viscous dietary fibres may for example be native, or modified, e.g. hydrolyzed.

In even further embodiments, additional components may be added to the rye extract of the invention, such as minerals.

In another embodiment the invention relates to a method of lowering the GI and/or improving the GP and/or GP2 of a food composition comprising the step of addition of above described rye extract or supplemented rye extract of the invention to a food composition.

In one aspect the invention relates to a food composition comprising a rye extract of the invention or supplemented rye extract of the invention.

In a further embodiment the present invention relates to a food composition comprising an effective amount of the rye extract or supplemented rye extract of the invention. By effective amount is meant herein an amount of the rye extract or supplemented rye extract of the invention sufficient to lower the GI of the food composition by for example 70%, 65%, 60%, 50%, 40%, 30%, 50%-20%, 50%-30%. 40%-20%, 20%, 20%-10%, 10% or 5% where the food composition of the invention is compared to the food composition without supplementation by the rye extract or supplemented rye extract of the invention.

An effective amount may also be described in terms of percent increase in GP and/or GP2, where an effective amount may be for example an increase in GP and/or GP2 of 5%, 10%, 10-20%, 20%, 30%, 40%, 30-40%, 40-40%, 45-50%, 50%, 50-60%, 70%, 80%, 90%, 100%, 100-150%, 190%, 200%.

An effective amount may also be described in terms of percent increase in the rye extract carbohydrates in a food composition of the invention relative to the food composition without the addition of the rye extract or supplemented rye extract of the invention. An effective amount may be for example increase of 100%, 150%, 200%, 70%, 60%, 50%, 40%, 30%, 20% 10% or for example 100%-150%, 50%-100% 75%-100%, 20%-70%, 20%-50%, 25%, 25 to 75%, 75% to 100%.

The food compositions of the invention may have a GI of below 85, such as 84, 83, 82, 81, 80, 79, such as 66, such as 65, below 60, below 55, such as 54, 53, 52, 51. The food compositions of the invention may have a GP of above 20, above 30, such as 40, 50, 60, 70, 80, 90, 100, 100-120, 110, 110-140, 120, 120-150, 160, 170, 180, 190, 200.

The food compositions of the invention may have a weight ratio of rye extract or supplemented rye extract to food composition of from 0.1% to 100%, such as 5%, 20%, 30%, 40%, 50%, 69%, 70%, 80%, 20-30%, 30-40%, 40-60%, 70-90%, 75-95%, 80-85%. Thus in one embodiment, the food composition of the invention consists of the rye extract or the supplemented rye extract.

In further embodiments the invention relates to food compositions, feeds, drinks, functional foods, functional feed, medicaments, nutraceuticals, nutritional supplements, medicaments and pharmaceuticals comprising the rye extract or supplemented rye extract of the invention. The invention also relates to the use of the rye extract according to the invention or the supplemented rye extract according the invention or a food composition according to the invention in the manufacture of a food, a feed, a drink, a dosage form, a functional food, a functional feed, a pharmaceutical or a medicament.

The rye extract, supplemented rye extract or food composition of the invention may appear as a solid, a semi-solid such as a cream or paste, a gel, a liquid, a dispersion, a suspension or an emulsion, a powder for dissolution, or in any desired form. The composition may appear, for example, in the form of all kinds of food, feed, drink, functional food and functional feed, e.g. as flakes, bars, breads, cookies and biscuits, as juice, soft drink, oat suspension, soya milk, dairy products such as yoghurt, chocolate, jam, pudding and other dairy desserts, spreadable products, frozen desserts and ice-cream, malt drink, coffee, tea, sport drink, meal replacement, gruel, porridge, ready to eat meals, infant formula, baby food; in the form of a pharmaceutical composition and medicament, e.g. as a powder, an aggregate, a granulate, a tablet, a coated tablet, a lozenge, a capsule, a drink, a syrup, a composition for tube feeding, for enteral intake, for oral administration and for enteral administration.

In one example the food composition of the invention is a baked product, such as a bread, bun, biscuit, cake, crisp bread. Said baked product may beside the rye extract comprised in the supplemented rye extract of the invention further comprise flour from grains or cereals such as wheat, rye, barley, oats, rice, sorghum, millet, and quinoa.

In one example the food composition is a bread. Rye flour with good baking qualities lack the components of the rye extract of the invention and the beneficial effect which said extract imparts. Thus, one embodiment of the invention relates to a rye bread, which further comprises the rye extract or supplemented rye extract of the invention wherein said rye bread comprises rye flour, and wherein said rye extract or supplemented rye extract comprises a rye extract from one or more of rye varieties selected from Vicello and Picasso or a combination thereof. In one alternative bread said rye flour does not comprise flour from either Vicello or Picasso.

The food compositions according to the invention can be prepared by conventional techniques, including, for example, mixing the rye extract or supplemented rye extract of the invention with at least one edible or pharmaceutically acceptable component, or, alternatively, by mixing the rye extract or the food supplement, together with one or more of said edible or pharmaceutically acceptable components, optionally followed by bringing the obtained food composition in a desired form by conventional techniques.

In another embodiment, the rye extract, supplemented rye extract or the food composition of the invention may be in the form of a dosage unit, being a food composition presented in a form and/or package which allows its direct use by the consumer, for example in the form of tablets, granules or powder preferably packed in a unit dose.

In a further embodiment, the present invention relates to the use of a rye extract, supplemented rye extract or a food composition according to the present invention in the manufacture of a food, a feed, a drink, a dosage form, a functional food, a functional feed, a pharmaceutical or a medicament.

In a further embodiment, the present invention relates to the use of a rye extract, supplemented rye extract or food composition according to the present invention for modifying the glycaemic response to the meal in humans or mammals that are healthy, at risk for, or suffer from one or more diseases related to insulin regulation.

In a further aspect of the present invention relates to the use of the rye extract, supplemented rye extract or food composition according to the invention, for treating, controlling or preventing diseases or conditions associated with metabolic or insulin resistance syndrome. In a further aspect the invention relates to the rye extract or the supplemented rye extract or the food product of the invention for use in treating, controlling or preventing diseases or conditions associated with insulin regulation.

Examples of disease or conditions associated with insulin regulation include metabolic syndrome, insulin resistance, diabetes, obesity, or symptoms and conditions associated with these disorders. The use may be for weight control, improved appetite regulation, increased satiety etc. The use may be for example by oral and/or enteral intake or administration, for example of a dose in conjunction with meals.

In a further embodiment the present invention relates to a method of treatment, comprising administering to a subject in need of such treatment an effective amount of a rye extract, supplemented rye extract or food composition of the invention in a suitable dosage form. Preferably, the doses are taken together with, or shortly before, e.g. 15 minutes before, the main meals, e.g. in the morning, at noon, and in the evening.

In a further aspect the present invention relates to the use of the rye extract, supplemented rye extract or food composition according to the invention to elicit glucagon-like peptide 1 (GLP-1) and/or gastric inhibitory polypeptide (GIP) secretion, or the use of the rye extract, supplemented rye extract or food composition of the invention in the manufacture of a medicament to elicit GLP-1 and/or GIP secretion.

In a further aspect the present invention relates to the use of the rye extract, supplemented rye extract or food composition according to the invention to stabilise the levels of ghrelin in a human being or mammal.

Yet a further embodiment of the present invention is the use of the rye extract, supplemented rye extract or food composition of the invention in the promotion of satiety, control of appetite or weight loss or in the maintenance of body weight.

The invention further provides a method of improving the bodily appearance of a mammal which comprises orally administering to said mammal a rye extract, supplemented rye extract or food composition of the invention, in a dosage effective to influence the glucose metabolism, and repeating said dose until a cosmetically beneficial loss of body weight has occurred.

### EXAMPLES

### Example 1

### Bread

### Bread Recipe

1020 g water
348 g white wheat flour
1044 g whole grain rye flour
24 g dry yeast

### 12 g NaCl

125 g of Rye extract

The dough was mixed in a mixing bowl for 6 min and was proofed in room temperature for 30 min. The dough was divided into pieces of 1 kg each and placed in a bread making tin, followed by a second proofing for 60 min in room temperature. Baking was performed at 250° C. for 40 min.

### Example 2

### Yoghurt

2.5 dl yoghurt containing a probiotic culture was blended with the rye extract of the invention yielding approximately 2 g carbohydrates per dl yoghurt. The carbohydrates present corresponded to those in Table 1.

### Example 3

### Preparation of Extract

Milled rye (kernels or whole straw) is dispersed in water or phosphate buffer and heated to 37° C. and stirred for 48 hours. This procedure is followed by sonication of the solution. The solution is heated to 95° C. and stirred for 30 minutes. Ethanol is added to the digest to reach 60% concentration and the solution is left to precipitate. The solution is then filtrated and the water phase is concentrated using drying.

### Analysis of Extract.

The amount of carbohydrates in the extract is analysed by LC using AOAC Official method 2001.03. Analysis of raffinose, stachyose and fructans was done using HPLC. Analysis of arabinoxylans and arabinogalactans was done by gas chromatography. Resistant starch and beta-glucans were analysed by enzymatic in vitro analysis.

### Example 4

### Composition of a Food Product Comprising a Rye Extract of the Invention

| | | % dry weight in |
|---|---|---|
| Type of carbohydrate | Description | starting material |
| Raffinose | | 0.08 |
| Stachyose | | 0.002 |
| Fructan | DP 3-15 | 3.2-4 |
| Fructan | DP 16-60 | 0.01-10 |
| Arabinoxylan | DP 3-100 | 0.002-10 |
| Arabinogalactan | DP 3-100 | 0.002-1.5 |
| Resistant starch | | 0.002-4 |
| Beta-glucanes (rye) | | 0.002-2.5 |

### Example 5

### Comparison of Different Rye Varieties

### Test Products

Five wholegrain breads made from different rye varieties grown in Sweden, were included in the study together with one rye bread baked from a commercial Swedish wholegrain rye blend and one white wheat (endosperm) reference bread (WWB). The whole grain rye breads were made from Vicello, Picasso, Kaskelott, Amilo and Evolo rye, respectively. Rye kernels of the commercial blend and Vicello were provided by Lilla Harrie mills (Kävlinge, Sweden); the other rye varieties were provided by Lantmännen SW Seed AB (Svalöv, Sweden) who also milled all rye kernels in the study. Commercial white wheat flour was obtained from Kungsörnen AB (Järna, Sweden). Dry yeast was acquired from Jästbolaget AB (Sollentuna, Sweden).

The WWB was made according to Rosén et al (Nutrition Journal 2011). The rye breads were made from 3000 g of whole grain rye flour, 1000 g of white wheat flour, 2700 g water, 50 g dry yeast and 40 g NaCl and baked at Pågen bakery, Malmö, Sweden. The doughs were mixed for 10 min and were proofed in room temperature for 40 min. The doughs were then divided into pieces of 1000 g each, placed in baking tins and subjected to a second proofing (37° C., 77% humidity) for 60 minutes. Baking was initiated at 250° C., the temperature was immediately lowered to 200° C. and the breads were baked for 35 minutes.

The WWB was left to cool for 1 hour and the rye breads for 22-24 hours under cover. Thereafter, the crust was removed and the breads were sliced and wrapped in aluminium foil in portion sizes, put into plastic bags and stored in a freezer (−20° C.) until use.

### Chemical Analysis of the Test Products

Prior to analyses, the bread samples were air dried and milled to pass through a 0.5 mm screen (Cyclotec, Tecator, Höganäs, Sweden). The available starch content was determined according to Holm et al. (Starch, 1986). Insoluble and soluble dietary fibre was determined with a gravimetric, enzymatic method described by Asp et al. (J Agri Food Chem 1983). Protein content was determined using an elemental analyser (FlashEA 1112, Thermo Fisher Scientific Inc., Waltham, Mass., USA). The nutritional compositions of the test products are presented in Table 2.

### Meal Study

### Test Subjects

20 healthy non-smoking volunteers (10 men and 10 women) aged 21-37 y (26.7±0.9; mean±SEM) with normal body mass indices (22.2±0.39 kg/m2; mean±SEM), and without drug therapy participated in the study. All subjects had normal fasting plasma glucose concentrations (5.2±0.03 mM; mean±SEM). The subjects were recruited in January-September 2010 and the study was performed from April to October, 2010. All test subjects gave their informed consent and were aware of the possibility of withdrawing from the study at any time they desired. Approval of the study was obtained by the Ethics Committee in Lund, Sweden (reference number 556/2008).

### Study Design

The products were provided as breakfast meals on 7 different occasions in random order, with approximately 1 wk between each test. The day before the experiment, the bread was taken from the freezer and thawed at ambient temperature, still wrapped in aluminium foil and in the plastic bag. The subjects were instructed to eat a standardized meal in the evening (21:00-22:00) prior to the test, consisting of a few slices of white wheat bread, and to avoid eating and drinking anything but small amounts of water until the start of the test on the following morning. In addition, the subjects were also told to avoid alcohol and excessive physical exercise the day before each test. The subjects reported to the laboratory at 0745 on the test day. A peripheral venous catheter (BD Venflon, Becton Dickinson, Helsingborg, Sweden) was inserted into an antecubital vein to be used for plasma sampling and fasting blood samples were taken prior to the meal. All products contributed with 50 g available starch and were served with 250 ml of tap water and the test subjects were instructed to finish the test meals within 14 minutes.

### Physiological Parameters

Capillary blood samples were taken for analysis of plasma glucose (p-glucose) and venous blood samples were drawn for the analysis of serum insulin (s-insulin) before the meal (0 min) and at 15, 30, 45, 60, 90, 120, 150 and 180 min after commencing the breakfast. In addition, the subjects were asked to fill in their subjective feeling of fullness, hunger and desire to eat, respectively, using a 100 mm Visual Analogue Scale (VAS). P-glucose concentrations were determined in capillary whole blood using a p-glucose analyser (Glucose 201+, Hemocue, Ängelholm). Serum was left to set for 30 minutes and then centrifuged for 12 min (1300*g, 4° C.). Serum was then immediately frozen at −20° C. until analysis. The s-insulin measurement was performed on an integrated immunoassay analyser (CODA Open Microplate System; Bio-rad Laboratories, Hercules, Calif., USA) by using an enzyme immunoassay kit (Mercodia AB, Uppsala, Sweden).

### Calculations and Statistical Methods

The data are expressed as means±SEM. One subject was excluded from the analysis of the Kaskelott rye bread breakfast due to having a cold on that particular test day. The data for Kaskelott is therefore analysed with n=19. Four subjects had missing values in the recordings of subjective satiety after the commercial rye bread, causing skewed data. Therefore, these subjects were excluded from all statistical analysis of subjective satiety. The total and net incremental areas under the glucose, insulin and appetite curves (tAUC and iAUC) were calculated for each subject and test meal, using the trapezoid model. The glycaemic index (GI) and insulinaemic index (II) were calculated using the iAUC (0-120 min) for p-glucose and s-insulin, respectively, with WWB as a reference (20). Glucose and insulin incremental peaks (iPeak) were calculated as maximum postprandial increase from baseline (fasting). The glycaemic profile (GP), defined as the duration of the glucose curve divided with the glucose iPeak, was calculated (11). GP2 was calculated in the same way as GP, but the duration was divided with the squared glucose iPeak, to increase the influence of the highest measured postprandial glucose concentration.

Time×treatment interactions were analysed using a mixed model (PROC MIXED in SAS release 8, SAS Institute Inc., Cary, N.C.) with repeated measures and an autoregressive covariance structure. Subjects were modelled as a random variable and the corresponding baseline (fasting values) was modelled as covariate. The data was analysed using a mixed model analysis of covariance (ANCOVA) with subjects as a random variable and corresponding baseline (fasting values) as a covariate (MINITAB, release 16, (Minitab Inc., State College, Pa.). Differences between groups were identified using Tukey's multiple comparison tests. In the cases of unevenly distributed residuals (tested with Anderson-Darling test), Box Cox transformation were performed on the data prior to the analysis. Correlation analysis was conducted to evaluate the relation among dependent measures with the use of Spearman's partial correlation coefficients controlling for subjects and corresponding baseline values (two-tailed test, SPSS software, version 19; SPSS Inc., Chicago, Ill., USA). p<0.05 was considered statistically significant.

### Results

### Glucose Responses

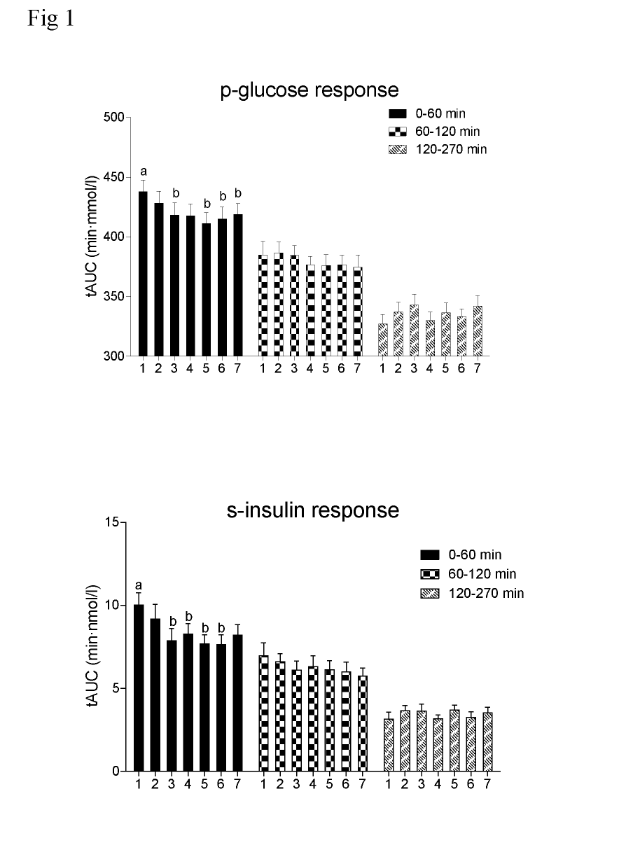

Vicello and Picasso rye displayed significantly lower GI values (79 and 80, respectively) than WWB (Table 3). The glucose iPeak was significantly lower than that of WWB following all rye breads, except those made of the commercial blend and Kaskelott rye, respectively. All rye breads but those made from commercial blend and Evolo displayed significantly lower early glucose response (tAUC 0-60 min) than WWB (FIG. 1). The GP values were not significantly different between any of the products, but the GP2 was significantly higher for the Vicello and Picasso breads compared to WWB. Furthermore, the GP2 of Vicello rye was significantly higher than that of the commercial rye blend. No time×treatment interaction was found (0-180 min, p=0.23).

The Visello and Picasso rye were described by a significantly higher GP2 than WWB, indicating a more beneficial and well regulated course of glycemia (see also Definitions for explanation of GP2). The GP2 was also well correlated with both a lowered insulin iPeak and II.

### Insulin Responses

All rye breads, with the exception of commercial rye blend and Evolo rye had a significantly lower II than WWB (Table 3). The early insulin response, expressed as tAUC 0-60 min and insulin iPeak was significantly lower for all rye breads except the ones made from commercial blend and Kaskelott rye (FIG. 1). No time×treatment interaction was found (0-180 min, p=0.24).

### Subjective Satiety

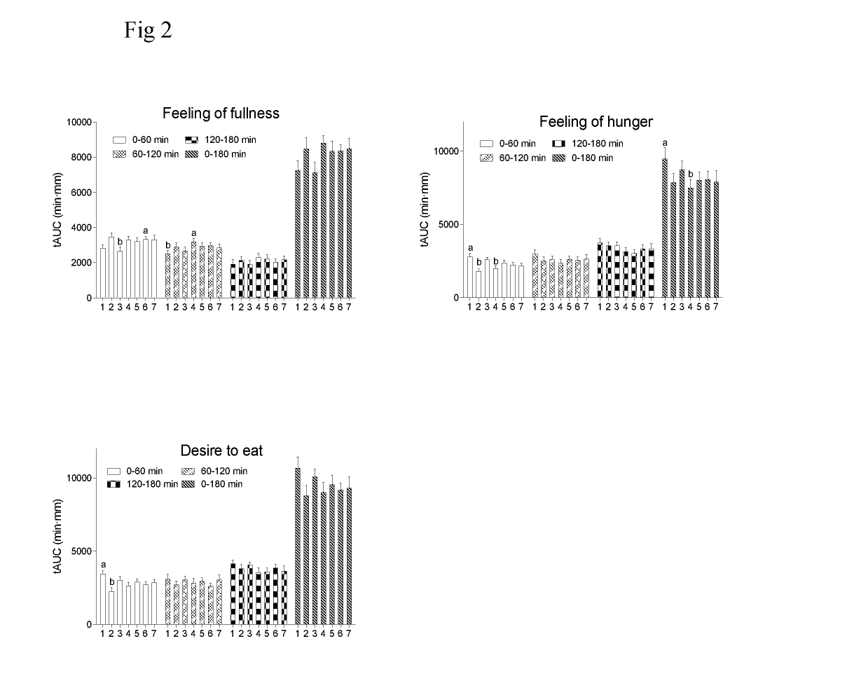

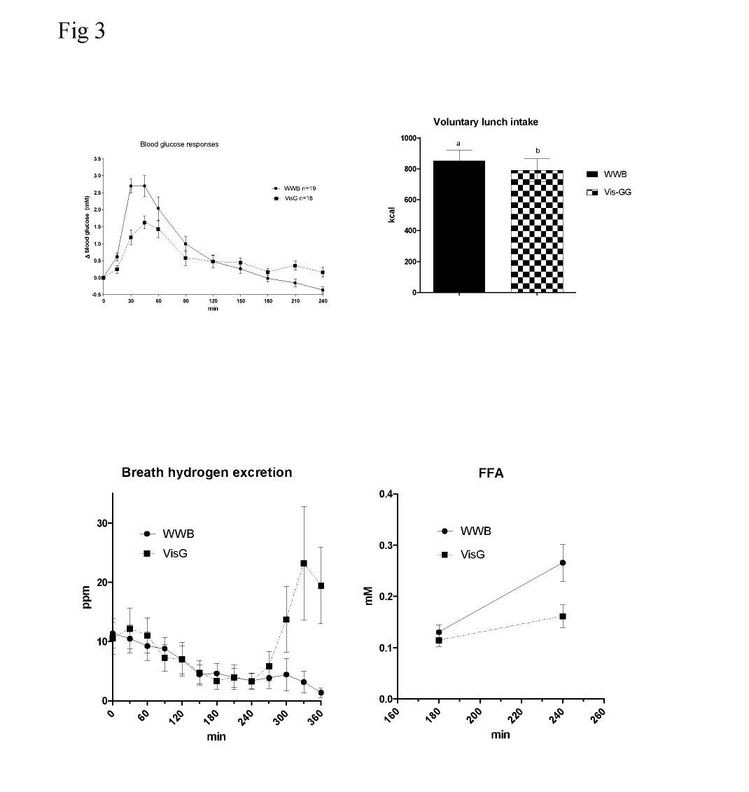

In the early postprandial phase (tAUC 0-60 min), the subjective feeling of fullness was significantly higher following Vicello rye bread compared to Amilo rye bread (FIG. 2). The subjective feeling of hunger and desire to eat was significantly lower after the commercial rye blend compared to after the WWB. Also Evolo rye bread induced significantly lower feeling of hunger compared to WWB in the early postprandial phase. Evolo rye induced a higher feeling of fullness compared to WWB during 60-120 min after breakfast (tAUC) and also a significantly lower feeling of hunger when analysing the entire study period (tAUC 0-180 min). No time×treatment interaction was found for feeling of fullness, hunger or desire to eat (0-180 min, p=0.48, 0.91, 0.75, respectively).

### Correlations

Correlations between postprandial glucose, insulin and subjective satiety are presented in Table 4. The GI, GP and GP2 were all significantly related to the insulin iPeak and II. However, the GP and GP2, respectively, showed a stronger relation to the insulin iPeak than did the GI.

A low glucose iPeak and a high GP2 was related to an improved subjective satiety in the late postprandial phase (tAUC 120-180 min and/or at 180 min). Also, the late postprandial desire to eat (180 min) was positively correlated to the insulin iPeak. A high insulin iPeak was related to a lower subjective feeling of hunger in the early postprandial phase (tAUC 0-60 min).

A high content of insoluble fibres in the rye breads was related to an improved subjective satiety in the early postprandial phase (tAUC 0-60 min) (fullness, hunger and desire to eat, r=0.24, −0.28, −0.43, respectively, p<0.05). A higher insoluble fibre content in the rye breads also lowered the desire to eat in the 60-120 min postprandial phase (r=−0.21, p<0.05).

### Example 6

### Combination of Rye with Soluble Fibre

One test bread product and one reference bread were served as breakfast meals at two different occasions. Four hours after the respective test breakfast meal, a second meal buffet style lunch was served. Nineteen healthy volunteers of both genders (normal BMI, 20-42 y) participated. The test bread product contained medium molecular weight guar gum (G) at a level of 10% (dry matter), combined with whole grain Visello (Vis) rye flour from KWS LOCHOW GMBH, Bergen, Germany. The portion contained 50 g available starch. Bread made from white wheat flour (WWB) was used as reference. The glycaemic index (GI) of the bread using WWB as reference (GI=100) was significantly lower for VisG (60.5). The iPeak (highest level) for glucose was 3.2 for WWB and 1.7 the test product. The glycaemic profile (GP), calculated as the duration of the curve divided by the iPeak, was significantly improved for VisG (86) compared to WWB (51). At 240 min (directly before lunch) the levels of free fatty acids (FFA) were significantly lower after eating VisG compared to WWB (0.16 mM and 0.27 mM, respectively). Breath hydrogen (H₂) was measured during each test day as a marker of colonic fermentation. The hydrogen excretion at the time of the time of lunch (AUC 300-360 min) was substantially increased after having VisG (1140 min*ppm) for breakfast, compared to WWB (82 min*ppm). The voluntary energy intake at lunch was 7.2% lower after VisG (791 kcal) compared to WWB (852 kcal, p=0.029).

The elevated level of H₂ after lunch strengthens our hypothesis that rye, in particular the variety Visello, causes a very early fermentation. In addition, the level of FFA was decreased after VisG compared to WWB. This is interesting since suppressed levels of FFA have been associated with increased insulin sensitivity.

### Example 7

### Rye Extraction with Water

26 g Visello flour was mixed with 100 ml water in a household blender for 2 min, and then transferred into the tubes which were incubated at room temperature (21° C.) for 2 h. The slurry was centrifuged for 20 min at 3000 rpm (ALC refrigerated centrifuge PK 130R, Sweden) and 100 ml water was mixed with the pellet before washing in the household blender for 2 min prior to being centrifuged again. The supernatants (water-soluble fraction) from the two centrifugations were poured together. At the same time, the pellet which was the insoluble fraction were freeze-dried and milled (CYCLOTEC 1093 Sample Mill) into powder through a 1.5 mm screen.

### Example 8

### Composition of Rye Extract

A rye extract was produced by allowing a starting material to ferment. Table 5 column “Starting material” indicates the percentage of each component prior to fermentation, while the column “fermented extract” indicates the percentage of the same component after fermentation. For example, Fructan DP=1-10 was present in the starting material, but was degraded during fermentation so that only 20% of the original amount was left after fermentation. The table also shows that some components are not present in the starting material (eg Arabinoxylan oligosaccharides (AXOS) DP=2-10), but are formed during the fermentation.

The fermentation may for example be step k.) according to the method of the invention. The extraction using different amounts of ethanol may affect the resulting amounts of each component in the extract. The extraction may for example be according to step g.) of the method of the invention. In table 5 the percentage composition of the rye extract after different ethanol concentrations are presented.

### TABLES

TABLE 2
Composition of the breakfast products.

| | Portion | Water | | Insoluble | Soluble |
|---|---|---|---|---|---|
| Meals | size | content | Protein | fibre | fibre |
| g/portion | | | | | |
| WWB | 125.92 | 61.65 | 6.68 | 2.43 | 0.37 |
| Amilo | 154.85 | 72.84 | 9.92 | 8.95 | 3.57 |
| Evolo | 152.58 | 70.21 | 9.01 | 9.29 | 3.99 |
| Kaskelott | 154.06 | 71.42 | 8.93 | 10.04 | 3.77 |
| Picasso | 153.88 | 72.67 | 8.98 | 9.71 | 3.71 |
| Vicello | 148.89 | 68.59 | 7.48 | 10.09 | 2.97 |
| Commercial blend | 157.62 | 74.16 | 7.60 | 10.58 | 3.32 |

n = 2 (Available starch and proteins),
n = 3 (fiber content).

TABLE 3
Glucose and insulin responses after the breakfast products.

| | GP | GP² | Glucose | GI | Insulin | II |
|---|---|---|---|---|---|---|
| Meals | min/mM | min/mM² | ΔmM | % | ΔnM | % |
| Vicello | 59.5 ± 9.5^{a} | 31.9 ± 13. | 2.9 ± 0.2^{b} | 79 ± 8.0^{b} | 0.165 ± 0. | 73.7 ± 7.9^{b} |
| Amilo | 54.2 ± 7.4^{a} | 26.9 ± 10. | 3.1 ± 0.2^{b} | 90 ± 8.4^{ab} | 0.180 ± 0. | 80.3 ± 7.2^{b} |
| Picasso | 52.3 ± 4.7^{a} | 26.7 ± 7.9^{a} | 2.9 ± 0.2^{b} | 80 ± 8.4^{b} | 0.175 ± 0. | 81.5 ± 9.6^{b} |
| Evolo | 52.8 ± 4.6^{a} | 19.3 ± 2.6^{a} | 3.2 ± 0.2^{b} | 92 ± 8.1^{ab} | 0.179 ± 0. | 84.8 ± 7.4^{a} |
| Commercial | 48.2 ± 6.7^{a} | 20.0 ± 5.2^{b} | 3.4 ± 0.2^{ab} | 95 ± 8.3^{ab} | 0.207 ± 0. | 92.5 ± 9.8^{a} |
| Kaskelott | 47.9 ± 4.1^{a} | 16.6 ± 2.3^{a} | 3.2 ± 0.2^{ab} | 88.4 ± 8.5^{a} | 0.186 ± 0. | 81.5 ± 7.1^{b} |
| WWB | 41.5 ± 2.8^{a} | 12.5 ± 1.5^{c} | 3.8 ± 0.2^{a} | 100 ± 0.0^{a} | 0.237 ± 0. | 100 ± 0^{a} |

Values are means ± SEM.
N = 20 for p-glucose and s-insulin responses (n = 19 for Kaskelott rye).
Products not sharing the same letters were significantly different.
P < 0.05.
ANCOVA followed by Tukey's test.

TABLE 4
Correlations between postprandial glucose, insulin and subjective satiety.

| | | | | Fullness | Fullness | | Hunger | | Desire to |
|---|---|---|---|---|---|---|---|---|---|
| | | | insulin | tAUC | tAUC | Hunger | tAUC | Desire to | eat tAUC |
| | | II | iPeak | 0-60 min | 120-180 | 180 min | 120-180 | eat 180 min | 120-180 |
| Glucose | r = | 0.51 | 0.52 | −0.13 | −0.21 | 0.35 | 0.28 | 0.34 | 0.33 |
| iPeak | p = | <0.001 | <0.001 | 0.201 | 0.031 | <0.001 | 0.003 | <0.001 | 0.001 |
| GP | r = | −0.32 | −0.43 | 0.13 | 0.14 | −0.25 | −0.14 | −0.31 | −0.22 |
| | p = | <0.001 | <0.001 | 0.194 | 0.145 | 0.010 | 0.163 | 0.001 | 0.021 |
| GP² | r = | −0.45 | −0.54 | 0.16 | 0.22 | −0.24 | −0.16 | −0.30 | −0.24 |
| | p = | <0.001 | <0.001 | 0.108 | 0.024 | 0.015 | 0.110 | 0.002 | 0.012 |
| GI | r = | 0.43 | 0.33 | 0.04 | −0.07 | 0.23 | 0.12 | 0.24 | 0.17 |
| | p = | <0.001 | <0.001 | 0.715 | 0.494 | 0.018 | 0.237 | 0.014 | 0.081 |
| II | r = | | 0.82 | −0.10 | −0.02 | 0.11 | 0.08 | 0.22 | 0.17 |
| | p = | | <0.001 | 0.302 | 0.808 | 0.260 | 0.390 | 0.022 | 0.077 |
| insulin | r = | 0.82 | | −0.20 | 0.01 | 0.15 | 0.03 | 0.27 | 0.18 |
| iPeak | p = | <0.001 | | 0.039 | 0.888 | 0.115 | 0.725 | 0.004 | 0.057 |

Spearman's partial correlation coefficients controlling for subjects and corresponding baseline values (two-tailed test). Significant correlations are shown in bold text.
n = 20 for glucose and insulin and 16 for subjective satiety.
For Kaskelott n = n − 1. Significant correlations are shown in bold text.

TABLE 5
Composition of extract

| | | | | Extraction | |
|---|---|---|---|---|---|
| | | | Extraction | with | |
| | | Fermented extract | with 0% EtOH | 40% EtOH | Extraction with |
| Dietary fibre component | Starting | % of | (water) (% of | (% of | 100% EtOH |
| (g/100 g dry weight basis | material | starting material | extract) | extract) | (% of extract) |
| Fructan DP = 1-10 | 100 | 20 | 4 | 4 | 4 |
| Fructan DP = 10-20 | 100 | 90 | 19 | 19 | 23 |
| Arabinoxylan DP >10 | 100 | 70 | 7 | 5 | 1 |
| Arabinoxylan oligosaccharides | 0 | 30 | 6 | 6 | 7 |
| (AXOS) DP = 2-10 | | | | | |
| Non-cellulosic B-glucans DP >100 | 100 | 50 | 11 | 10 | 8 |
| Mannoproteins | 0 | 100 | 21 | 21 | 14 |
| Raffinose DP = 3-5 | 100 | 50 | 11 | 11 | 14 |
| Mellobiose DP = 2 | 0 | 100 | 21 | 21 | 28 |

1. A method of making a rye extract comprising the steps of:
a.) providing a rye material, such as rye kernels and/or plant material from rye such as whole straw;
b.) milling said rye material;
c.) dispersing said milled rye material in a liquid;
d.) incubating said dispersion of step c.);
e.) optionally solubilizing and/or disintegrating aggregates in the dispersion from step d.);
f.) optionally heating the solubilized dispersion from step e.);
g.) optionally adding an alcohol suitable for human consumption, such as ethanol to reach a concentration of from 20% to 100% and allowing precipitation to take place;
h.) optionally filtering away the precipitate from step g.);
i.) recovering the liquid phase, for example by centrifuging and recovering the liquid supernatant; and
j.) optionally concentrating the liquid phase from i.).

2. The method according to claim 1 wherein a fermentation step k.) is inserted after step c.), wherein one or more probiotic is added.

3. The rye extract obtainable by the method of claim 1.

4. The rye extract obtained by the method of claim 2.

5. A rye extract according to claim 1 comprising Fructan (DP 1-10), Fructan (DP=10-20), Arabinoxylans (DP>10), Non-cellulosic beta-glucans (DP>100) and Raffinose (DP=3-5), as well as Arabinoxylan oligosaccharides (DP=2-10), mannoproteins and Mellobiose (DP=2).

6. The method according to claim 1 wherein the rye material is selected from the group consisting of rye variety Visello and Picasso or a combination thereof.

7. A supplemented rye extract comprising the rye extract of claim 3 further comprising soluble viscous dietary fibre.

8. The supplemented rye extract according to claim 7 wherein the soluble viscous dietary fibre is selected from the group consisting of one or more of agar, alginates, carubin, pectin, beta-glucan, carrageenans, furcellaran, psyllium, mucilages and gums, alfalfa, clover, fenugreek, tamarind flour, pectin, scleroglucan, mannoglucans, konjac gum, xanthan gum, guar gum, gum tragacanth, arabic gum, karaya gum, gum ghatti, gellan gum and other related sterculia gum.

9. A food composition comprising the rye extract claim 3.

10. A method of using the rye extract according to claim 3 in the manufacture of a food, a feed, a drink, a dosage form, a functional food, a functional feed, a pharmaceutical or a medicament.

11. The rye extract according to claim 3 for use in treating, controlling or preventing diseases or conditions associated with insulin regulation.

12. A method of improving the bodily appearance of a mammal which comprises orally administering to said mammal a rye extract according to claim 3.
